(19) United States
(12) Patent Application Publication
(10) Pub. No.: US 2016/0184323 A1
(43) Pub. Date: Jun. 30, 2016

(54) PHARMACEUTICAL COMPOSITIONS FOR INTRAOCULAR ADMINISTRATION AND METHODS FOR FABRICATING THEREOF

(71) Applicant: Imprimis Pharmaceuticals, Inc., San Diego, CA (US)

(72) Inventors: William F. Wiley, Chagrin Falls, OH (US); Richard Dilzer, Long Valley, NJ (US)

(21) Appl. No.: 15/061,488

(22) Filed: Mar. 4, 2016

Related U.S. Application Data
(62) Division of application No. 14/461,242, filed on Aug. 15, 2014.
Application No. 14/461,242 is a continuation-in-part of application No. 14/227,819, filed on Mar. 27, 2014.
(60) Provisional application No. 61/958,170, filed on Jul. 22, 2013.

(51) Int. Cl. A61K 31/573; A61F 9/008; A61K 47/34; A61K 31/4709; A61K 9/00

(52) U.S. Cl. CPC A61K 31/573; A61K 31/4709; A61K 9/0048; A61K 47/34; A61F 9/008; A61F 2009/00893

(57) ABSTRACT

Pharmaceutical compositions for intraocular injection are described, the compositions consisting essentially of a therapeutically effective quantity of an anti-bacterial agent (such as moxifloxacin), a therapeutically effective quantity of an anti-inflammatory agent (such as prednisolone), at least one pharmaceutically acceptable excipient and a pharmaceutically acceptable carrier. Methods for fabricating the compositions and using them for intraocular injections are also described.

### CROSS REFERENCE TO RELATED APPLICATIONS

This is a divisional of U.S. patent application Ser. No. 14/461,242, filed Aug. 15, 2014, now pending, which is a continuation-in-part under 35 U.S.C. §120 of U.S. patent application Ser. No. 14/227,819, filed on Mar. 27, 2014, which in turn claims priority under 35 U.S.C. §119(e) to U.S. Provisional Application No. 61/958,170, filed on Jul. 22, 2013, the entire content of each of which is hereby incorporated by reference.

### FIELD OF THE INVENTION

The present invention relates generally to the field of ophthalmology and more specifically to injectable ophthalmological compositions having anti-bacterial and anti-inflammatory properties, and to methods of preparing such compositions.

### BACKGROUND

In ophthalmological treatments and procedures, e.g., cataract surgery, pre- and post-operative eye drops are frequently used by the patients to eliminate or alleviate negative post-surgery complications such as infections, inflammation, and tissue edema. It has been reported that as many as 8% of all ocular surgery patients may suffer from infections, including the potentially catastrophic endophthalmitis, and various negative sight threatening side effects after surgery, such as inflammatory uveitis, corneal edema, and cystoid macular edema. Typically, the topical postoperative medications are prescribed for at-home use starting before and then after cataract surgery, and are typically self-administered, unless requiring a caregiver or family assistance.

These ophthalmic medication drops include anti-inflammatory and antibiotic agents and are highly effective, but require strict adherence to the treatment regimens, which is often difficult for many patients (with physical limitations or aversions to eyelid touching and manipulation) and is frequently expensive (well over $200 per procedure), causing patients' dissatisfaction. It is desirable to have an alternative procedure that would permit avoiding the necessity of the use of such post-surgery medications to save the associated post-operative trouble and expenses.

One such alternative procedure includes the intraoperative intravitreal injection by an atraumatic transzonular route that can achieve patient outcomes that are as good as, or better than, the current at-home eye drop regimen, removing the issues of compliance and medication administration accuracy. This patent specification discloses pharmaceutical compositions suitable for intraoperative ocular injections that can achieve such positive patient outcomes, and methods of fabricating and administering the same.

### SUMMARY

According to one embodiment of the invention, a pharmaceutical composition for intraocular injection is provided, the composition comprising a therapeutic component consisting essentially of a therapeutically effective quantity of an anti-bacterial agent and a therapeutically effective quantity of an anti-inflammatory agent, and at least one pharmaceutically acceptable excipient and/or a pharmaceutically acceptable carrier that are suitable for intraocular injection.

According to another embodiment of the invention, an anti-bacterial agent described herein can be a compound selected from the group of quinolone (including a fluorinated quinolone), e.g., moxifloxacin, and pharmaceutically acceptable salts, hydrates, solvates or N-oxides thereof.

According to yet another embodiment of the invention, an anti-inflammatory agent described herein can be a corticosteroid, e.g., triamcinolone, and pharmaceutically acceptable salts, hydrates, solvates, ethers, esters, acetals and ketals thereof.

According to another embodiment of the invention, the pharmaceutical compositions described herein may further include a solubilizing and suspending agent such as non-ionic polyoxyethylene-polyoxypropylene block copolymer, e.g., Poloxamer 407®.

According to other embodiments of the invention, the pharmaceutical compositions described herein may be intravitreally transzonularly injected into a mammalian subject as a part of the process of treatment of a variety of ophthalmological diseases, conditions or pathologies associated with intraocular surgery, such as cataracts, retinal and glaucoma disease.

### DETAILED DESCRIPTION

A. Terms and Definitions

Unless specific definitions are provided, the nomenclatures utilized in connection with, and the laboratory procedures and techniques of analytical chemistry, synthetic organic and inorganic chemistry described herein, are those known in the art. Standard chemical symbols are used interchangeably with the full names represented by such symbols. Thus, for example, the terms “hydrogen” and “H” are understood to have identical meaning. Standard techniques may be used for chemical syntheses, chemical analyses, formulating compositions and testing them. The foregoing techniques and procedures can be generally performed according to conventional methods well known in the art.

It is to be understood that both the foregoing general description and the following detailed description are exemplary and explanatory only and are not restrictive of the invention claimed. As used herein, the use of the singular includes the plural unless specifically stated otherwise. The section headings used herein are for organizational purposes only and are not to be construed as limiting the subject matter described.

As used herein, “or” means “and/or” unless stated otherwise. Furthermore, use of the term “including” as well as other forms, such as “includes,” and “included,” is not limiting.

“About” as used herein means that a number referred to as “about” comprises the recited number plus or minus 1-10% of that recited number. For example, “about” 100 degrees can mean 95-105 degrees or as few as 99-101 degrees depending on the context. Whenever it appears herein, a numerical range such as “1 to 20” refers to each integer in the given range; i.e., meaning only 1, only 2, only 3, etc., up to and including only 20.

The term “pharmaceutical composition” is defined as a chemical or a biological compound or substance, or a mixture or combination of two or more such compounds or substances, intended for use in the medical diagnosis, cure, treatment, or prevention of disease or pathology.

The term “intraocular injection” refers to an injection that is administered by entering the eyeball of the patient. The term “peri-ocular injection” refers to an injection that is administered behind the eye but outside the eye wall. The term “transzonular” refers to an injection administered through the ciliary zonule which is a series of fibers connecting the ciliary body and lens of the eye.

The term “intravitreal” refers to an injection administered through an eye of the patient, directly into the inner cavity of the eye.

The term “intraoperative” is defined as an action occurring or carried during, or in the course of, surgery.

The term “keratomileusis” refers to a surgical procedure whereby the refractive state of the cornea is improved. This procedure can be performed as a laser-assisted in situ surgery also known as “LASIK.” Other corneal refractive surgical procedures that are encompassed by the term “keratomileusis” include, without limitations, photorefractive keratectomy (PRK), laser-assisted sub-epithelial keratectomy (LASEK), corneal ring segments, corneal cross linking, refractive corneal inlays (e.g., “raindrop”, “Kamra”) and corneal lenticular surgery (“SMILE”).

The terms “anti-bacterial” and “antibiotic” are broadly covered by the term “anti-microbial” and are used herein interchangeably, refer to substances or compounds that destroy bacteria and/or viruses and/or inhibit the growth thereof via any mechanism or route.

The term “anti-inflammatory” refers to substances or compounds that counteract or suppress inflammation via any mechanism or route.

The term “quinolone” for the purposes of this application refers to a genus of anti-bacterial compounds that are derivatives of benzopyridine and in some embodiments include fluorine atom, such as in the following structure (“fluoroquinolone”):

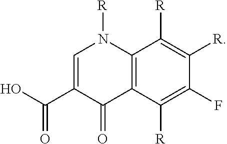

The terms “corticosteroid” and closely related “glucocorticoid” are defined as compounds belonging to a sub-genus of steroids that are derivatives of corticosterone, the latter having the chemical structure:

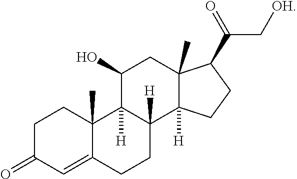

The term “salt” refers to an ionic compound which is a product of the neutralization reaction of an acid and a base.

The terms “solvate” and “hydrate” are used herein to indicate that a compound or a substance is physically or chemically associated with a solvent for “solvates” such as water (for “hydrates”) .

The term “ether” refers to a chemical compound containing the structure R—O—R₁, where two organic fragments R and R₁ are connected via oxygen.

The term “ester” refers to a chemical compound containing the ester group R—O—C(O)—R₁, connecting two organic fragments R and R₁.

The terms “acetal” and “ketal” refer to a chemical compound containing the functional group R—C(R₁)(OR₂)₂, where R and R₂ are organic fragments and R₁ is hydrogen atom (for acetals), and is inclusive of “hemiacetals” where one R₂ (but not the other) is hydrogen atom; or where none of R, R₁ and R₂ is a hydrogen atom and each is an organic fragment (for ketals).

The term “carrier” refers to a substance that serves as a vehicle for improving the efficiency of delivery and the effectiveness of a pharmaceutical composition.

The term “excipient” refers to a pharmacologically inactive substance that is formulated in combination with the pharmacologically active ingredient of pharmaceutical composition and is inclusive of bulking agents, fillers, diluents and products used for facilitating drug absorption or solubility or for other pharmacokinetic considerations.

The term “therapeutically effective amount” is defined as the amount of the compound or pharmaceutical composition that will elicit the biological or medical response of a tissue, system, animal or human that is being sought by the researcher, medical doctor or other clinician.

The term “pharmaceutically acceptable” is defined as a carrier, whether diluent or excipient, that is compatible with the other ingredients of the formulation and not deleterious to the recipient thereof.

The terms “administration of a composition” or “administering a composition” is defined to include an act of providing a compound of the invention or pharmaceutical composition to the subject in need of treatment.
B. Embodiments of the Invention

According to embodiments of the present invention, pharmaceutical compositions intended to prevent and/or treat inflammation and/or infections are provided. The compositions include an active component comprising, consisting essentially of, or consisting of a therapeutically effective quantity of an anti-bacterial agent (i.e., an antibiotic) and a therapeutically effective quantity of an anti-inflammatory agent (e.g., a corticosteroid). In some embodiments, the pharmaceutical compositions can be used for intraocular injections. In other embodiments the pharmaceutical compositions can be used for intra-articular or intra-lesional use. The compositions further include one or several pharmaceutically acceptable excipient(s) and one or several pharmaceutically acceptable carrier(s).

The concentration of the anti-bacterial agent in the pharmaceutical composition may be between about 0.01 mg/mL and about 50.0 mg/mL, such as between about 0.5 mg/mL and about 10 mg/mL, for example, about 1.0 mg/mL. The concentration of the anti-inflammatory agent in the pharmaceutical composition may be between about 0.1 mg/mL and about 100.0 mg/mL, such as between about 5.0 mg/mL and about 50.0 mg/mL, for example, about 15.0 mg/mL.

According to further embodiments, the anti-bacterial agent to be employed in the active component of the composition may be selected from the group of quinolones, including fluoroquinolones, and suitable derivatives of the same, such as pharmaceutically acceptable salts, hydrates or solvates thereof. In one embodiment, fluoroquinolone that may be so employed is moxifloxacin (chemically, 1-cyclopropyl-7-[(1S,6S)-2,8-diazabicyclo-[4.3.0]non-8-yl]-6-fluoro-8-methoxy-4-oxo-quinoline-3-carboxylic acid), which is available, e.g., under trade name Avelox® from Bayer Healthcare Corp. of Wayne, N.J., and under other trade names from other suppliers such as Alcon Corp. and Bristol-Myers Squibb Co. and has the following chemical structure:

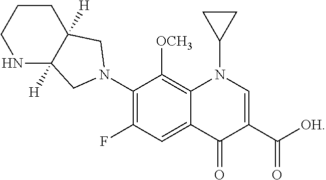 [SMILES: COc1c(N2C[C@@H]3CCCN[C@@H]3C2)c(F)cc2c(=O)c(C(=O)O)cn(C3CC3)c12]

A non-limiting example of a possible alternative fluoroquinolone antibiotic that may be used instead of, or in combination with, moxifloxacin is gatifloxacin. In some embodiments one or several glycopeptide antibiotic(s), or a combination of some or all of them, may be optionally used as a part of the anti-bacterial agent, in combination with moxifloxacin. One example of such an acceptable additional glycopeptide antibiotic is vancomycin which can be introduced into the pharmaceutical composition at a concentration between about 1 mg/mL and about 100.0 mg/mL, such as between about 5.0 mg/mL and about 50.0 mg/mL, for example, about 10.0 mg/mL. Vancomycin is available under the trade name Vancocin® from Eli Lilly & Co. of Indianapolis, Ind. Other acceptable additional glycopeptide antibiotics that may be used include teicoplanin, telavancin, decaplanin, ramoplanin, gentamicin, tobramycin, amikacin, cefuroxime, polymyxin B sulfate, and trimethoprim.

According to further embodiments, the anti-inflammatory agent to be employed in the active component of the composition may be selected from the group of corticosteroids, such as derivatives of corticosterone, and pharmaceutically acceptable salts, hydrates, solvates, ethers, esters, acetals and ketals thereof. For example, a product obtained as a result of a chemically reasonable substitution of any hydrogen and/or hydroxyl group in the molecule of corticosterone may be used. In one embodiment, a corticosteroid that can be so utilized is triamcinolone (chemically, (11β,16α)-9-fluoro-11,16,17,21-tetrahydroxypregna-1,4-diene-3,20-dione) having the following chemical formula:

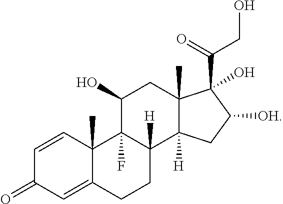

In another embodiment, a corticosteroid that can be so utilized is triamcinolone acetonide (chemically, (4aS,4bR,5S,6aS,6bS,9aR,10aS,10bS)-4b-fluoro-6b-glycoloyl-5-hydroxy-4a,6a,8,8-tetramethyl-4a,4b,5,6,6a,6b,9a,10,10a,10b,11,12-dodecahydro-2H-naphtho[2′,1′:4,5]indeno[1,2-d][1,3]dioxol-2-one) which is a ketal derivative of triamcinolone available, e.g., under the trade name Kenalog® from Bristol-Myers Squibb Co. of Princeton, N.J., and under other trade names from other suppliers, and having the following chemical formula:

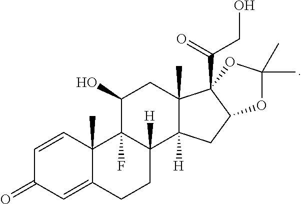

Other corticosteroids, or a combination of some or all of them, may be used instead of all or a portion of triamcinolone and/or of all or a portion of triamcinolone acetonide. Some non-limiting examples of such acceptable other corticosteroids or glucocorticoids include triamcinolone diacetate, triamcinolone benetonide, triamcinolone furetonide, triamcinolone hexacetonide, betamethasone acetate, dexamethasone, fluorometholone and fluocinolone acetonide, prednisone, prednisolone, methylprednisone, corticol, cortisone, fluorocortisone, deoxycorticosterone acetate, aldosterone, budesonide and derivatives, analogs or combinations thereof.

Some of these corticosteroids, e.g., without limitation, prednisone, prednisolone, dexamethasone, or methylprednisone are considered particularly suitable in methods for performing a keratomileusis or corneal refractive surgery (e.g., LASIK surgery) described below in more detail. Those having ordinary skill in the art of ophthalmology or pharmacy will determine which corticosteroids are to be used in a specific surgical procedure to be performed.

As mentioned above, the pharmaceutical composition that is the subject matter of the instant application may further optionally include one or several pharmaceutically acceptable excipient(s). Those having ordinary skill in the art will be able to select the suitable excipient(s). It is worth mentioning that when moxifloxacin is used in pharmaceutical formulations, it is often difficult to obtain a stable suspension of another product (e.g., a corticosteroid such as triamcinolone acetonide) that is present in the same formulation and that needs to be in a form of a stable suspension. Without being bound by any particular scientific theory, such difficulties in obtaining the stable suspension are believed to be caused by moxifloxacin's tendency to deactivate many suspending agents resulting in unacceptable coagulation, clumping and flocculation. As a result, normal delivery through a typical 27-29 gage cannula is often difficult or even impossible.

Therefore, it is desirable to select an excipient that is stable in the presence of moxifloxacin and can, therefore, be used as a solubilizing and suspending agent to ensure that the corticosteroid such as triamcinolone acetonide safely forms a stable suspension even when moxifloxacin is also present in the same formulation. Numerous attempts by others to produce a stable moxifloxacin/triamcinolone acetonide pharmaceutical composition suitable for intraocular injection have not been successful.

In some embodiments, an excipient that can be used as a solubilizing and stabilizing agent to overcome the above-described difficulties and thus to obtain a stable suspension of the corticosteroid such as triamcinolone acetonide may be a non-ionic polyoxyethylene-polyoxypropylene block copolymer having the following general structure:

HO—(CH₂—CH₂—O)ₓ—(C₃H₆—O)_{y}—(CH₂—CH₂—O)ₓ—H,

wherein x is an integer having the value of at least 8 and y is an integer having the value of at least 38.

If a non-ionic polyoxyethylene-polyoxypropylene block copolymer is used as a solubilizing and stabilizing agent in the pharmaceutical compositions of the instant invention, its contents in the overall composition may be between about 0.01 mass % and about 10.0 mass % such as between about 1.0 mass % and about 8 mass %, for example, about 5.0 mass %.

One non-limiting example of a specific non-ionic polyoxyethylene-polyoxypropylene block copolymer that can be used as a solubilizing and stabilizing agent in the pharmaceutical compositions of the instant invention is the product known under the trade name Poloxamer 407® (poly(ethylene glycol)-block-poly(propylene glycol)-block-poly(ethylene glycol)) available from Sigma-Aldrich Corp. of St. Louis, Mo., with the molecular weight of the polyoxypropylene portion of about 4,000 Daltons, about a 70% polyoxyethylene content, the overall molecular weight of between about 9,840 Daltons and about 14,600 Daltons and having the following chemical structure:

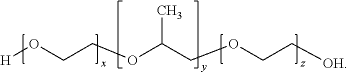

Non-limiting examples of some other excipients and carriers that may be used in preparing in the pharmaceutical compositions of the instant invention include polysorbate (an emulsifier), edetate calcium disodium (EDTA, a chelating agent), hydrochloric acid (the pH adjuster) and sterile water.

According to further embodiments, methods for fabricating the above-described pharmaceutical compositions are provided. A one-batch formulation method may be used, where the components of the pharmaceutical formulation can be combined in single container; the components may be added to the container simultaneously or consecutively.

In one exemplary, non-limiting procedure, a quantity of an anti-bacterial agent such as moxifloxacin may be placed into a mixing container followed by adding a quantity of sterile water and hydrochloric acid to obtain a slightly acidic mixture (e.g., having pH of about 6.5) which is stirred until a clear solution is obtained. In case of moxifloxacin/HCl system, the solution is stable, allowing the formulation to remain closed system thus preventing contamination and the loss of sterility.

Next, a quantity of corticosteroid such as micronized triamcinolone acetonide, a quantity of Poloxamer 407®, a quantity of edetate calcium disodium and a quantity of polysorbate 80 may be all added to be combined in the same container with the already prepared moxifloxacin/HCl solution and stirred together (e.g., by spinning) for a period of time, e.g., about 6 hours, until a homogenous suspension has been obtained. The resulting suspension may then be transferred into single dose vials, capped, sealed, autoclaved and shaken until cool. Finally, a complete testing for sterility and the presence of endotoxin may be performed on the product according to commonly used methods known to those having ordinary skill in the art.

Pharmaceutical compositions prepared as described above can be used to prevent complications that may arise after ophthalmic surgical operations and procedure. For example, the formulations can be used during any intraocular surgery, such as cataract surgery, planned vitrectomy or glaucoma procedures, to prevent or at least substantially reduce the risk of post-surgery complications, such as the development of endophthalmitis or cystoid macular edema (CME), without having the patient use pre- or post-operative topical ophthalmic drops. Individuals with evidence of endophthalmitis from prior surgical procedures or traumatic ocular penetration will benefit from concurrent injection of these formulations to sterilize infection and reduce damaging inflammation.

Pharmaceutical formulations described herein can be delivered via intraocular intravitreal injection which can be transzonular, or, if desired not transzonular. Intraocular intravitreal injection of this formulation, whether done via transzonular or via direct pars plana (trans-scleral) injection, delivers potent broad spectrum antibiotics directly into the suppurative tissue without requiring the urgent compounding of multiple individual medications or multiple individual injections into the eye.

Typically, a pharmaceutical composition described above will be intraocularly administered to a mammalian subject (e.g., humans, cats, dogs, other pets, domestic, wild or farm animals) in need of emergent, urgent or planned ophthalmic surgery treatment. The effect achieved by such use of pharmaceutical composition described above may last up to four weeks. The composition is to be injected intravitreally and trans-zonularly using methods and techniques known to those having ordinary skilled in the art of ophthalmology. In some embodiments, the injection can be intraoperative.

Typically, the delivery through a typical 27 gauge cannula can be employed utilizing a 1 mL TB syringe, with attention to re-suspending the formulation using momentary flicks and shake just prior to injection. The medicinal volume (i.e., dosage) required of this formulation varies based on the type of intraocular procedure, the degree of postoperative inflammation induced or anticipated, the risk assessment for postoperative infection, and anatomic considerations regarding the available volume for the injection being added to a closed intraocular space.

It is worth mentioning that while intracameral (that is, anterior chamber) injections are within the scope of the instant invention such injections instead of posterior chamber (intravitreal) injection may not be satisfactory in some cases, as the suspension clogs the trabecular meshwork and aggravates intraocular drainage, resulting in an intraocular pressure rise postoperative. This is avoided with intravitreal injection, in addition to retaining the formulation components into the protein matrix of the vitreous of a greater duration. Anterior chamber wash out occurs over hours (antibiotic in solution) and days (steroid in suspension), while intravitreal injection is retained for weeks.

In alternative embodiments, if desired or necessary the formulations may also be delivered in the form of eye drops or eye sprays, as well as via subconjunctival injection, intraocular intracameral injection, sub-tenon injection, intra-articular injection or intra-lesional injection, particularly, in, but not limited to, some cases when necessary to deliver additional medication when local ocular inflammation and extra-ocular infection need suppression. Intravitreal delivery of steroid has historically been used to treat clinically significant cystoid macular edema (CME); the application of this formulation into the vitreous during routine intraocular procedures brings more aggressive prophylaxis against CME occurrence. Additionally, the suspension of this formulation is useful for staining vitreous during planned and unplanned vitrectomies, improving visualization of this otherwise transparent intraocular tissue, improving vitrectomy outcomes and reducing complications resulting from inadequate or tractional vitreous removal. In still further embodiments, there is also envisioned intra-canalicular delivery, i.e., delivery via a lacrimal canaliculus implant. In yet other embodiments, the formulation may be also delivered via anterior chamber injection or capsular bag placement of medication. A solution could be also added to the irrigating solution that is used during cataract surgery which could allow for the bottles, tubing, etc. to become “sterilized” during surgery. Intracorneal delivery through laser created corneal channels that could hold medication can be also used, if desired.

In some further alternative embodiments, instead of delivering the above-described compositions comprising both anti-bacterial and anti-inflammatory agents, consecutive injections may be used instead, if desired. For example, triamcinolone or prednisolone may be injected first, immediately followed by the injection of moxifloxacin or vice versa.

In still further embodiments, the pharmaceutical applications described hereinabove may be used before or after performing corneal refractive surgery or a keratomileusis surgery such as LASIK surgery. To illustrate, a pharmaceutical composition to be used for these purposes may include any corticosteroid and any anti-bacterial agent described above, to be selected by the skilled practitioner. To further exemplify but not to unduly limit, prednisone, prednisolone or methylprednisone may be chosen as the former and moxifloxacin as the latter. As a further non-limiting illustration, the formulations used in conjunction with a keratomileusis surgery may have the concentration of the anti-bacterial agent such as moxifloxacin between about 0.01 mg/mL and about 50.0 mg/mL, such as between about 0.5 mg/mL and about 10 mg/mL, for example, about 1.0 mg/mL; and the concentration of the corticosteroid such as prednisone between about 0.1 mg/mL and about 100.0 mg/mL, such as between about 5.0 mg/mL and about 50.0 mg/mL, for example, about 15.0 mg/mL.

It will be understood by those having ordinary skill in the art that the specific dose level and frequency of dosage for any particular patient may be varied and will depend upon a variety of factors including the activity of the specific compound employed, the metabolic stability and length of action of that compound, the age, body weight, general health, gender, diet, and the severity of the particular ophthalmological condition being treated.

In additional embodiments, pharmaceutical kits are provided. The kit includes a sealed container approved for the storage of pharmaceutical compositions, the container containing one of the above-described pharmaceutical compositions. An instruction for the use of the composition and the information about the composition are to be included in the kit.

The following examples are provided to further elucidate the advantages and features of the present invention, but are not intended to limit the scope of the invention. The examples are for the illustrative purposes only. USP pharmaceutical grade products were used in preparing the formulations described below.
C. Examples

### Example 1

### Preparing a Pharmaceutical Composition

A pharmaceutical composition was prepared as described below. The following products were used in the amounts and concentrations specified:
(a) about 1.5 g of triamcinolone acetonide, at a concentration of about 15.0 mg/mL;
(b) about 0.1 g of moxifloxacin hydrochloride, at a concentration of about 1.0 mg/mL;
(c) about 1 mL of polysorbate 80, at a concentration of about 1.0 mass %;
(d) about 0.2 g of edetate calcium disodium, at a concentration of about 0.2 mass %;
(e) about 1 g of Poloxamer 407®, at a concentration of about 1.0 mass %;
(f) hydrochloric acid, to adjust pH to about 6.5; and
(g) about 100.0 mL of sterile water for injection.

Moxifloxacin hydrochloride was placed into a de-pyrogenated beaker with a spin bar. Sterile water for injection was added to about ⅓ of the volume of the beaker. While spinning, moxifloxacin was dissolved by adding hydrochloric acid until a clear solution having the final pH of about 6.5 was obtained.

The solution was combined with micronized triamcinolone acetonide, Poloxamer 407®, edetate calcium disodium and polysorbate 80 and allowed to spin for about 6 hours until a hydrated and homogenous suspension was obtained.

The suspension was transferred into de-pyrogenated, single dose vials (2 mL size), capped and sealed, followed by autoclaving and shaking the vials until cool. Complete sterility and endotoxin testing was performed by an outside laboratory to ensure safety.

The formulation prepared as described above was tested for the particle sizes and their distribution. The results showed that very fine particles were obtained and the size distribution was quite uniform. Specifically, about 99% of all the particles had the diameter of 5 μM or less, where the sizes within the range between about 1 μM and 4 μM dominated and constituted about 82% of all particles. Just 0.1 to 0.2% of all the particles were large than about 10 μM in diameter.

The formulation prepared as described above was also tested for stability after 6 months of storage. After this period of storage no loss of potency was observed (as measured by HPLC); the formulation was visually stable at room temperature and readily re-suspended with gentle shaking with no increase of particle size or flocculation.

### Example 2

### Preparing a Pharmaceutical Composition Containing Vancomycin

A pharmaceutical composition was prepared as described in Example 1, supra. The composition was autoclaved and sonicated for about 60 minutes and about 96 mL of the composition were combined with about 4 mL of vancomycin at a concentration of about 250 mg/mL. The pH of the mixture was adjusted to about 6.0-6.5 using hydrochloric acid. The product was then transferred into vials (at about 1 mL plus 5 drops per vial) and frozen. The product has kept its stability and potency for at least six months.

### Example 3

### Using a Pharmaceutical Composition

A pharmaceutical composition fabricated as described in Example 1, supra, was administered to about 1,600 patients. To each, it was introduced using intravitreal transzonular injection. The injection was intraoperative. Only a very few patients, at the rate of about only 1 in 4,000, have developed any infection or suffered from other side effects that required further treatment, which is a substantial improvement over a typical rate of about 8% for the patients that did not receive the injection.

### Example 4

### Preparing a Pharmaceutical Composition Containing Prednisolone

A pharmaceutical composition was prepared as described below. The following products were used in the amounts specified:
(a) about 1.5 g of micronized prednisolone acetate;
(b) about 0.1 g of moxifloxacin hydrochloride;
(c) about 1 mL of an aqueous solution of polysorbate 80, at a concentration of about 1.0 mass %;
(d) about 0.2 g of edetate calcium disodium;
(e) about 1.2 g of Poloxamer 407®;
(f) hydrochloric acid, to adjust pH to about 6.5; and
(g) about 100.0 mL of sterile water for injection.

Moxifloxacin hydrochloride was placed into a de-pyrogenated beaker with a spin bar. Sterile water for injection was added to about ⅓ of the volume of the beaker. While spinning, moxifloxacin was dissolved by adding hydrochloric acid until a clear solution having the final pH of about 6.0 to 6.5 was obtained.

The solution was combined with micronized prednisolone acetate, Poloxamer 407®, edetate calcium disodium and polysorbate 80 and allowed to spin until a hydrated and homogenous product was obtained. It is expected that the particle sizes and their distribution are similar to those described in Example 1, above. The product was then transferred into de-pyrogenated, single dose vials (about 1 mL of the product in 3 mL size vial), capped and sealed, followed by autoclaving, shaking and sonicating the vials for about 1 hour.

In a second experiment, another prednisolone-based composition was prepared in exactly the same way, except that the quantity of micronized prednisolone acetate was about 1.0 g (instead of about 1.5 g) and the quantity of moxifloxacin hydrochloride was about 0.5 g (instead of 0.1 g).

Prednisolone based composition obtained as described in this Example can then be administered to a patient by ordinarily skilled ophthalmologists as eye drops after performing a keratomileusis surgery such as LASIK surgery, e.g., as follow-up care.

Although the invention has been described with reference to the above examples, it will be understood that modifications and variations are encompassed within the spirit and scope of the invention. Accordingly, the invention is limited only by the following claims.

What is claimed is:

1. A method for treating an ophthalmological disease, condition or pathology in a mammalian subject in need of such treatment comprising delivering to the subject a composition comprising:
(a) a therapeutic component consisting essentially of:
(a1) a therapeutically effective quantity of an anti-bacterial agent independently selected from the group consisting of quinolone, a fluorinated quinolone and pharmaceutically acceptable salts, hydrates, solvates or N-oxides thereof; and
(a2) a therapeutically effective quantity of a corticosteroid independently selected from the group consisting of prednisone, prednisolone, methylprednisone, corticol, cortisone, fluorocortisone, deoxycorticosterone acetate, aldosterone, budesonide, derivatives or analogs thereof and pharmaceutically acceptable salts, hydrates, solvates, ethers, esters, acetals and ketals thereof;
(b) at least one pharmaceutically acceptable solubilizing and suspending agent selected from the group consisting of non-ionic polyoxyethylene-polyoxypropylene block copolymers; and
(c) optionally, a pharmaceutically acceptable carrier therefor,
wherein the method of delivery is selected from the group consisting of intravitreal injection, intraocular intracameral injection, intra-lesional injection, intra-articular injection, subconjunctival injection, sub-tenon injection, delivery via eye drops, delivery via spray and intra-canalicular delivery, to treat the ophthalmological disease, condition or pathology thereby.

2. The method of claim 1, wherein the mammalian subject is selected from the group consisting of a human, a cat, a dog, another pet, a wild animal and a farm animal.

3. The method of claim 1, wherein the method of delivery is delivery via eye drops.

4. A method for treating an ophthalmological disease, condition or pathology in a mammalian subject in need of such treatment comprising:
(a) performing a keratomileusis surgery on the subject; and
(b) administering to the subject a composition comprising:
a. a therapeutic component consisting essentially of:
a1. a therapeutically effective quantity of an anti-bacterial agent independently selected from the group consisting of quinolone, a fluorinated quinolone and pharmaceutically acceptable salts, hydrates, solvates or N-oxides thereof; and
a2. a therapeutically effective quantity of a corticosteroid independently selected from the group consisting of prednisone, prednisolone, methylprednisone, corticol, cortisone, fluorocortisone, deoxycorticosterone acetate, aldosterone, budesonide, derivatives or analogs thereof and pharmaceutically acceptable salts, hydrates, solvates, ethers, esters, acetals and ketals thereof;
b. at least one pharmaceutically acceptable solubilizing and suspending agent selected from the group consisting of non-ionic polyoxyethylene-polyoxypropylene block copolymers; and
c. optionally, a pharmaceutically acceptable carrier therefor.

5. The method of claim 4, wherein the keratomileusis surgery is selected from the group consisting of laser-assisted in situ surgery (LASIK), photorefractive keratectomy (PRK), laser-assisted sub-epithelial keratectomy (LASEK), corneal ring segments, corneal cross linking, refractive corneal inlays and corneal lenticular surgery.

6. The method of claim 5, wherein the keratomileusis surgery is LASIK surgery.

7. The method of claim 4, wherein the composition is administered via drops after the surgery.
